(19) United States
(12) Patent Application Publication
(10) Pub. No.: US 2022/0297137 A1
(43) Pub. Date: Sep. 22, 2022

(54) BIOLOGICAL COMPONENT SEPARATORS

(71) Applicant: HEWLETT-PACKARD DEVELOPMENT COMPANY, L.P., Spring, TX (US)

(72) Inventors: Hilary Ely, Corvallis, OR (US); Si-Iam J. CHOY, Corvallis, OR (US)

(73) Assignee: HEWLETT-PACKARD DEVELOPMENT COMPANY, L.P., Spring, TX (US)

(21) Appl. No.: 17/642,270

(22) PCT Filed: May 8, 2020
(86) PCT No.: PCT/US2020/032197

§ 371 Date: Mar. 11, 2022

Related U.S. Application Data
(63) Continuation of application No. PCT/US2019/058429, filed on Oct. 29, 2019.
Application No. PCT/US2019/058429 is a continuation of application No. PCT/US2019/058427, filed on Oct. 29, 2019.

(51) Int. Cl. B03C 1/03; G01N 35/00; B01L 3/00

(52) U.S. Cl. CPC B03C 1/03; G01N 35/0098; B01L 3/502; B01L 2200/0621; B01L 2200/0631; B01L 2200/0647; B01L 2400/0406; B03C 2201/18

(57) ABSTRACT

A biological component separator can include a vertically layered fluid column with a plurality of fluids positioned in fluid layers and a density-differential interface along which two fluids from the plurality of fluids are positioned. The biological component separator can also include a magnetic field generator positioned about the vertically layered fluid column and having multiple magnetic field profiles appliable across the vertically layered fluid column. The multiple magnetic field profiles can include a particle aggregating profile to concentrate magnetizing particles when present in the vertically layered fluid column and a particle sweeping profile to re-suspend and sweep the magnetizing particles when present across the vertically layered fluid column.

### BACKGROUND

In biomedical, chemical, and environmental testing, isolating a component of interest from a sample fluid can be useful. Such separations can permit analysis or amplification of a component of interest. As the quantity of available assays for components increases, so does the demand for the ability to isolate components of interest from sample fluids.

### BRIEF DESCRIPTION OF THE DRAWINGS

FIG. 1A illustrates an example biological component separator and a biological component separating system applying a first magnetic field using a particle aggregating profile;

FIG. 1B illustrates the example biological component separator and a biological component separation system of FIG. 1A applying a second magnetic field using a particle sweeping profile;

FIG. 2 illustrates an example biological component separator and a biological component separation system applying a first magnetic field using a particle aggregating profile and then a second magnetic field using a particle sweeping profile;

FIG. 3 illustrates an example biological component separator including both a multi-fluid density gradient portion and a capillary force gradient portion of a vertically layered fluid column, and further illustrates the use of multiple magnetic strengths to apply multiple magnetic fields thereto;

FIG. 4 illustrates an example biological component separator including both a multi-fluid density gradient portion and a capillary force gradient portion of a vertically layered fluid column, and further illustrates the use of magnetic shielding to apply multiple magnetic fields thereto;

FIG. 5 illustrates an example biological component separator and biological component separation system with an example sequential application of multiple magnetic fields including magnetic fields applied sequentially based on a particle aggregating profile and a particle sweeping profile; and

FIG. 6 is a flow diagram illustrating an example method of separating a biological component from a biological sample in accordance with examples of the present disclosure.

### DETAILED DESCRIPTION

In biological assays, a biological component can be intermixed with other components in a biological sample that can interfere with subsequent analysis. As used herein, the term “biological component” can refer to materials of various types, including proteins, cells, cell nuclei, nucleic acids, bacteria, viruses, or the like, that can be present in a biological sample. A “biological sample” can refer to a fluid obtained for analysis from a living or deceased organism. Isolating the biological component from other components of the biological sample can permit subsequent analysis without interference and can increase an accuracy of the subsequent analysis. In addition, isolating a biological component from other components in a biological sample can permit analysis of the biological component that would not be possible if the biological component remained in the biological sample. Many of the current isolation techniques can include repeatedly dispersing and re-aggregating samples. The repeated dispersing and re-aggregating can result in a loss of a quantity of the biological component in some instances. Furthermore, isolating a biological component with some of these techniques can be complex, time consuming, and labor intensive and can also result in less than maximum yields of the isolated biological component.

In accordance with examples of the present disclosure, a biological component separator can include a vertically layered fluid column with a plurality of fluids positioned in fluid layers and a density-differential interface along which two fluids from the plurality of fluids are positioned. The biological component separator can also include a magnetic field generator positioned about the vertically layered fluid column and having multiple magnetic field profiles appliable across the vertically layered fluid column. The multiple magnetic field profiles can include a particle aggregating profile to concentrate magnetizing particles when present in the vertically layered fluid column and a particle sweeping profile to re-suspend and sweep the magnetizing particles when present across the vertically layered fluid column. In one example, the density difference of the first fluid relative to the second fluid is from about 50 mg/mL to about 3 g/mL. In another example, the particle aggregating profile can include an incubation period, wherein during the incubation period, the magnetic particles are concentrated as they move across the density-differential interface along a z-axis of the vertically layered fluid column. In another example, the particle sweeping profile can include a dynamically changing magnetic field (which is inclusive of multiple dynamically changing magnetic fields) that causes magnetizing particles to move along a z-axis of the vertically layered fluid column and across the density-differential interface. The magnetic field generator can include, for example, one or multiple permanent magnets arranged to have a first spatial relationship relative to the vertically layered fluid column to apply the particle aggregating profile, and can also have a second spatial relationship relative to the vertically layered fluid column to apply the particle sweeping profile. The magnetic field generator can include one or multiple electrically induced magnetic elements. In this example, the particle aggregating profile and particle sweeping profile are different, which in this example can be a result of a different electrical profile applied to the one or multiple electrically induced magnetic elements.

In another example, a biological component separation system includes a vertically layered fluid column having a plurality of fluids positioned in fluid layers and a density-differential interface along which two fluids from the plurality of fluids are positioned, and magnetizing particles including particle surfaces to associate with a biological component of a biological fluid sample. The system also includes a magnetic field generator positioned about the vertically layered fluid column and having multiple magnetic field profiles appliable across the vertically layered fluid column. The multiple magnetic field profiles include a particle aggregating profile to concentrate the magnetizing particles and a particle sweeping profile to re-suspend and sweep the magnetizing particles across the vertically layered fluid column. In one example, system includes the biological fluid sample and the magnetizing particles can be associated with the biological component relative to secondary components in the biological fluid sample. In another example, the biological component can be a nucleic acid and the magnetizing particles can be surface-activated for positive isolation of the nucleic acid to separate the nucleic acid from secondary components. In another example, the biological component can be a secondary component that is other than a nucleic acid and the magnetizing particles are surface-activated for negative isolation to separate the nucleic acid from the secondary component. In other words, the magnetizing particles can attract the biological component to be processed when positive isolation is carried out. Alternatively, magnetizing beads can attract or bind to secondary components that are not of interest, so the magnetizing particles can be used to collect these materials to remove from the fluid that contains the biological component of interest. The fluids and/or magnetizing particles and/or surface chemistry can be selected to target and bind whatever components that are to be bound either. The particle aggregating profile can include, for example, an incubation period, and the particle sweeping profile can include a dynamically changing magnetic field. One or both of the particle aggregating profile or the particle sweeping profile can be used to cause magnetizing particles to move along a z-axis of the vertically layered fluid column and across the density-differential interface.

In another example, a method of separating a biological component from a biological fluid sample includes loading a biological fluid sample into a vertically layered fluid column. The biological fluid sample in the vertically layered fluid column includes a biological component associated with a surface of magnetizing particles and the vertically layered fluid column includes a plurality of fluids positioned in fluid layers and a density-differential interface along which two fluids from the plurality of fluids are positioned. The method further includes applying a first magnetic field having a particle aggregating profile across the vertically layered fluid column to aggregate the magnetizing particle with the biological component thereon, and applying a second magnetic field having a particle sweeping profile across the vertically layered fluid column to re-suspend and sweep the magnetizing particles with the biological component thereon across the density-differential interface. The method further includes magnetically moving the magnetizing particles across the density-differential interface using the first magnetic field, the second magnetic field, or a combination of both. In one example, the biological component may be a nucleic acid, and the biological sample includes secondary components that are not associated with the surface of the magnetizing particles, e.g., secondary components can be enzymes, cellular debris, lysing agents, buffers, or a combination thereof. In another example, the method can further include applying a third magnetic field having a particle aggregating profile that is different than the particle aggregating profile of the first magnetic field, and also applying a fourth magnetic field having a particle sweeping profile across that is different than the particle sweeping profile of the second magnetic field. The particle aggregating profile can include, for example, an incubation time ranging from 1 second to 5 minutes (where the magnetic field is held static so the particles can collect and aggregate). The sweeping profile can also include an incubation time ranging from 1 second to 5 minutes and/or may include a dynamically changing magnetic field (where the magnetic field is modified, such as by magnet movement, modified electromagnetic energy applied to electrically induced magnetic elements, etc.

It is noted that when discussing the biological component separator, the biological component separation system, or the method of separating a biological component from a biological sample, such discussions can be considered applicable to one another whether or not they are explicitly discussed in the context of that example. Thus, for example, when discussing a multi-fluid density gradient column or a multi-fluid density gradient portion of a vertically layered fluid column, such disclosure is also relevant to and directly supported in the context of the microfluidic biological component separation system, or the method of separating a biological component from a biological sample, and vice versa.

Terms used herein will have the ordinary meaning in the relevant technical field unless specified otherwise. In some instances, there are terms defined more specifically throughout the specification or included at the end of the present specification, and thus, these terms can have a meaning as described herein.

### Biological Component Separators and Systems

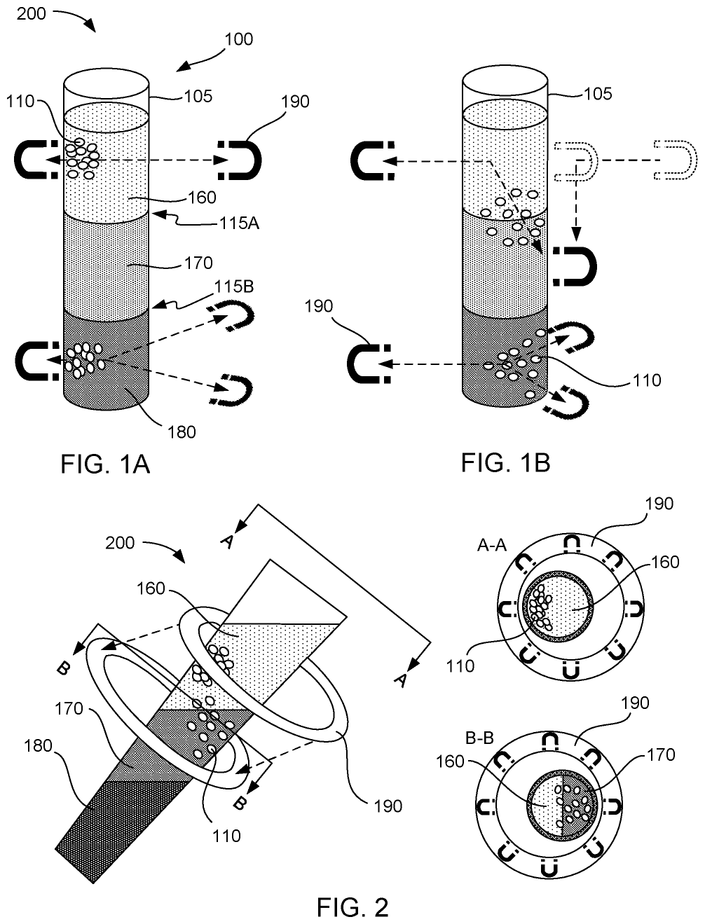

FIGS. 1A and B illustrate a biological component separator 100 that can be loaded with one or a multitude of magnetizing particles to provide a biological component separation system 200 (or “system”). Biological component separators and biological component separation systems are described herein together as the biological component separator can be part of the system (which includes magnetizing particles), and the biological component separator may be described more fully in the context of how it acts on the magnetizing particles.

In accordance with this, as shown in FIG. 1A and FIG. 1B, a biological component separator 100 can include a vertically layered fluid column that is shown as being supported by a vessel 105, and includes a first fluid 160 having a first fluid density, and a second fluid 170 having a second fluid density that is greater than the first fluid density. The second fluid in this example is separated and in direct contact beneath the first fluid at a density-differential interface 115A. In this particular example, there is also a third fluid 180 having a third fluid density that is greater than the second fluid density and is also separated from the second fluid at a density-differential interface 115B. The biological component separator also includes a magnetic field generator 190 (collective shown as five magnets in this example) positioned about the vertically layered fluid column. The magnets (which can be permanent magnets, electrically induced magnetic elements, or a combination of both), can provide multiple magnetic field profiles. The magnetic field generator can include, for example, one or multiple permanent magnets arranged to have a first spatial relationship relative to the vertically layered fluid column to apply the particle aggregating profile, and can also have a second spatial relationship relative to the vertically layered fluid column to apply the particle sweeping profile. The magnetic field generator can include one or multiple electrically induced magnetic elements. In this example, the particle aggregating profile and particle sweeping profile are different, which in this example can be a result of a different electrical profile applied to the one or multiple electrically induced magnetic elements.

It is noted that the term “first,” “second,” “third,” etc., are used for clarity in describing certain figures and for understanding the disclosure but should not be considered to be limiting. For example, fluid 180 could be referred to as the “first fluid,” or the “second fluid” or the “third fluid.” The other fluids could be similarly renamed without consequence to the scope of the disclosure. The mentioning of “first,” second,” “third,” etc., should be viewed in the context of the other layers in the biological component separator, biological component separation system, or method of separating a biological component, and not confused with other instances where the terms “first,” “second,” “third,” etc., may be used differently in another context, whether that be in the claims or specification disclosure. When the context would be clearer to refer to a “first fluid” as “fluid 160” or “fluid 170,” etc., than that naming convention may be alternatively used.

Referring now to FIGS. 1A and 1B, for example, this example shows the magnetic field generator 190 in a configuration with a particle aggregating profile to concentrate magnetizing particles 110 in the vertically layered fluid column. Concentrating magnetizing particles may include, for example, applying the magnetic field with an incubation time from 1 second to 5 minutes, from 1 second to 1 minute, from 2 seconds to 5 minutes, or from 2 seconds to 2 minutes, for example. Incubation time refers to a time frame where the magnetic field is held static to promote concentration or aggregation of the magnetizing particles. As shown more specifically in FIG. 1B, the same magnets are shown in a second position (as well as moving through multiple positions) which illustrates an example of application of a magnetic field(s) in accordance with a particle sweeping profile. This particle sweeping profile can be used to re-suspend and sweep the magnetizing particles from their aggregated state to a more diffuse state within the multi-fluid density gradient column when present across the vertically layered fluid column. In some instances, the particle aggregating profile can include an incubation period, wherein during the incubation period, the magnetic particles are concentrated as they move across the density-differential interface along a z-axis of the vertically layered fluid column. In another example, the particle sweeping profile can include a dynamically changing magnetic field that causes magnetizing particles to move along a z-axis of the vertically layered fluid column and across the density-differential interface, as shown in FIG. 1B.

In this and other examples herein, the first fluid 160, the second fluid 170, and the third fluid 180, and/or any other fluid layers can be arranged as any of a number of combinations of fluids, such as gas fluid, e.g., air, aqueous fluid, non-polar fluid, master mix fluid, reagent fluid, surface binding fluid, washing fluid, elution fluid, lysis fluid, etc. Fluids can, for example, contain a portion of the biological sample, e.g., blood, urine, saliva, transport media, etc. These or other fluids may be selected for use, and furthermore, these or other fluids may likewise be selected for use either above the first fluid or beneath the third fluid.

Referring now to FIG. 2, an alternative biological fluid separation system 200 is shown that includes a biological component separator 100 and magnetizing particles 110. A side view is shown where the biological component separator is positioned at an angle less than 90 degrees and greater than 45 degrees from horizontal. This is still considered to be a vertically layered fluid column, as the multi-fluid density gradient column includes density-differential interfaces 115 that are horizontal or about horizontal and the movement of the particles through the interfaces has a z-axis component. As shown in this FIG. is a top view taken at A-A as well as at B-B. As with FIGS. 1A and 1B, there is a first fluid 160, a second fluid 170, and a third fluid 180 of increasing fluid densities, and these are shown as being supported by a vessel 105. In this example, the magnetic field generator 190 is shown when applying a particle aggregating profile (shown from top view A-A) and then at a particle sweeping profile (shown from top view at B-B), which in this example can be a result of a different electrical profile applied to the one or multiple electrically induced magnetic elements. In this particular example, by moving the right side (as shown) of the magnet nearer to the multi-fluid density column beneath the density-differential interface, the magnetizing particles can be swept across the fluid and transverse the density-differential interface. The magnetic field generator is shown as including 8 individual magnets about a ring support but could be an annular magnet that is magnetized throughout or can have some other similar magnetizing element.

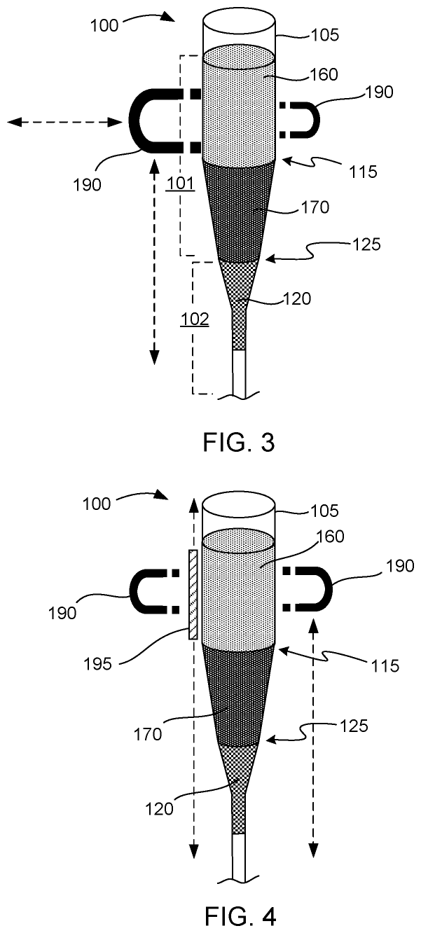

Regarding FIG. 3 and FIG. 4, two similar biological component separators 100 are shown. These can also be viewed in the context of biological component separation systems when magnetizing particles (not shown) are added thereto. In these two examples, the multi-fluid density gradient column shown, unlike that shown in FIGS. 1A, 1B, and 2, includes both a multi-fluid density gradient portion 101 and a capillary force-supported gradient portion 102. The two portions may be contained or supported by a vessel 105, which can include both an enlarged portion positioned about the vertically layered fluid column along the multi-fluid density gradient portion (where capillary forces are not used to provide the density-differential interface 115), and a narrower portion which may include a capillary tube positioned about the vertically layered fluid column along the capillary force gradient portion (where capillary forces do contribute to forming the capillary force-supported interface 125).

As an initial matter, the terms “density gradient” or “multi-fluid density gradient” can be as previously described. These terms can be used in various contexts herein but refers to the ability of multiple fluids to remain separated in layers due to their density difference (with denser fluids being positioned vertically lower along the column). Thus, there can be multiple fluids that are separated (such as in phases), but are still in direct contact at an fluid interface, referred to herein as a “density-differential interface,” which is descriptive of the interface being present as a result of the density difference.

On the other hand, the terms “capillary force” or “capillary force-supported gradient” refer to fluid interfaces that are not provided by their increasing density and their density difference, but rather, the fluids of immediately adjacent layers can have different densities, but less dense fluids can be positioned below denser fluids, and the reason these less dense fluid do not migrate upward is because they are constrained within a narrow fluidic channel due to the surface tension interaction between the fluid at the fluid interface, namely at the “capillary force-supported interface.” In accordance with the present disclosure, the fluid columns described herein that can be used include multi-fluid density fluid columns or column portions, but in some examples, may also include capillary force-supported gradient portions.

In this example (which could be applied to any of the examples herein), the multi-fluid density gradient portion includes two (or more) individual fluids of different densities. In this example, there is a first fluid 160 having a first fluid density and a second fluid 170 having a second fluid density that is greater than the first fluid density. The density difference between the first and the second fluid is sufficient so that the fluids remain separated at a (first) density-differential interface 115, even though the fluids are in direct contact with one another, e.g., the fluids are separated by their densities, not by a membrane or other artificial structure there between. However, as shown, the multi-fluid density gradient column, which can alternatively be referred to as a vertically layered fluid column, also includes a third fluid 120 that is less dense than the second fluid 170. Thus, in a more standard sized column, the third fluid would typically otherwise migrate up into or through the second fluid or even the fluids above if it is less dense than those fluids at other layers, destroying the interface between the second and third fluids and potentially at other interfaces. However, in the example shown, this is not the case. The third fluid is constrained by the cross-sectional size of the column structure (along the plane where the second fluid interfaces with the third fluid). Thus, the surface tension of the third fluid combined with the size constraint of the column at this interface in combination provide capillary force-supported interface 125, which promotes the second fluid and the third fluid remaining separated from one another. More specifically, the capillary force-supported interface can be contained within a fluidic channel having a cross-sectional dimension that is aligned with the capillary force-supported interface, and can be, for example, less than 2 mm, from 1 μm to 1 mm, from 1 mm to 1.75 mm, from 0.75 μm to 100 μm, or from 1 μm to 50 μm. This dimension can be an average diameter dimension, or for non-circular geometries, this average dimension can be the average cross-sectional dimension.

Example density differences of the first fluid relative to the second fluid (or any two fluids along the multi-fluid density gradient portion) can be from 50 mg/mL to 3 g/mL, from 100 mg/mL to 3 g/mL, from 500 mg/mL to 3 g/mL or from 1 g/mL to 3 g/mL, as is the case with the other similar density gradient columns or column portions. The “fluid density” can be measured conventionally by calibrating a scale to zero with the container thereon and then obtaining the mass of the fluid (liquid) in grams. The volume of the measure mass can then be determined using a graduated cylinder. The density is then calculated by dividing the mass by the volume to get the fluid density (g/mL).

With more specific detail related to the magnetic field generator 190, in addition to that shown by example previously, there are other possible magnet arrangements that can be suitable. For example, in FIG. 3, magnets of different strengths can be applied at different locations (as illustrated schematically by the size difference of the magnets). Thus, in one example, a weaker magnet can be affixed at or near a vessel wall to provide a first magnetic field with a particle aggregating profile, and a stronger magnet can be used to pull aggregated or concentrated magnetizing particles away from the weaker magnet to provide a second magnetic field in accordance with a particle sweeping profile. The stronger magnet and/or the weaker magnetic could then be repositioned for further use in the process or can be moved in a negative or positive z-direction cause the magnetizing particles to transverse the density-differential interface 115 or the capillary force-supported interface, for example. Alternatively, the stronger magnet and/or the weaker magnet could be electrically induced, and thus, the relative strengths could be modulated electrically rather than (or in addition to) physical movement of the magnets.

Regarding FIG. 4, the same structures and fluids are shown in example as well, except that rather than using multiple strengths of magnets for the magnetic field generator, the magnets could be permanent magnets and could be located at or near the vessel wall, and a moveable magnetic shield 195 could be positioned and repositioned to interrupt the magnetic field, thus provide a first or second magnetic field having a particle aggregating profile or a particle sweeping profile (or switching back and forth between the two profiles depending on where the magnetic shield is positioned. Furthermore, the magnets could likewise be moveable either laterally in the x- and/or y-axes as well as in the positive or negative z-direction. With a shield, the magnetic field could be greatly reduced or removed without moving the magnet, allowing the magnetic field of one magnet to interact with the magnetizing particles one at a time or primarily one at a time.

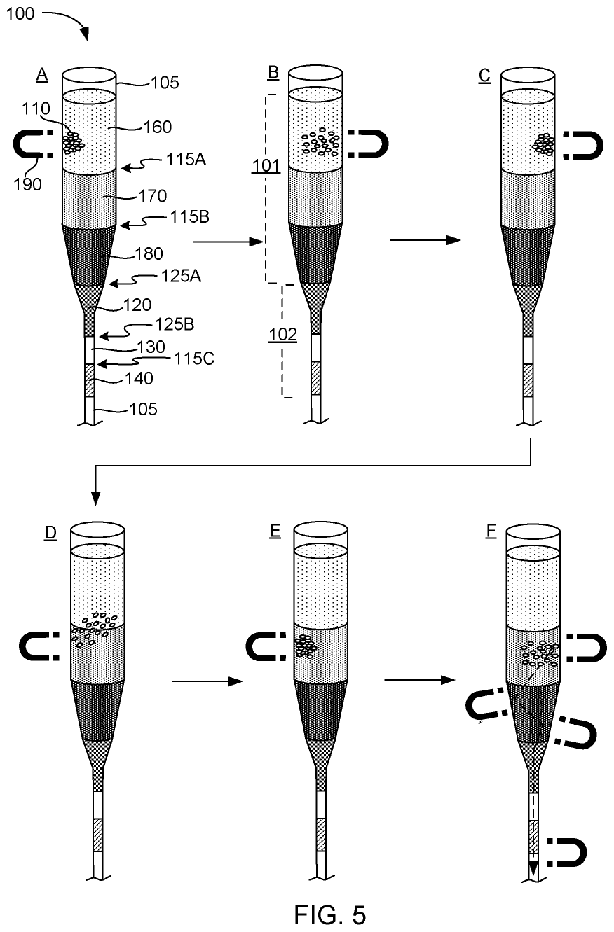

Referring now to FIG. 5, an example sequence is shown where multiple particle aggregating profiles are applied and multiple particle sweeping profiles are applied sequentially. This example is not considered to be limiting, but shows how the various magnetic fields applied according to the described profiles can act on the magnetizing particles as they are passed through the vertically layered fluid column (which in this example includes both a multi-fluid density gradient portion and a capillary force-supported gradient portion).

More specifically, in the present example, a vertically layered fluid column 100 can include two portions, namely a multi-fluid density gradient portion 101 and a capillary force gradient portion 102. The two portions may be contained or supported by a vessel 105, which can include both an enlarged portion positioned about the vertically layered fluid column along the multi-fluid density gradient portion (where capillary forces are not used to provide the density-differential interface shown at 115A and 115), and a narrower portion which may include a capillary tube positioned about the vertically layered fluid column along the capillary force gradient portion (where capillary forces do contribute to forming the capillary force-supported interface 125A and 125B).

The multi-fluid density gradient portion is shown in this example as including a first fluid 160, a second fluid 170, and a third fluid 180. The capillary force gradient portion includes fluid 120, fluid 130, and fluid 140. Fluid 120 may be, for example, an oil. Fluid 130 may be, for example, a gas such as an air gap, and fluid 140 may be, for example, an elution buffer or master mix fluid. Below fluid 140 represents further downstream processing that may occur. The fluids in the multi-fluid density gradient portion are separated by density-differential interfaces 115A and 115B, and the fluids in the capillary force gradient portion (including at the interface with fluid 180) are separated by capillary force-supported interfaces 125A and 125B. If fluid 130 is a gas and is less dense than the liquid layer at fluid 140, then these fluid can be separated due to their density and do not really benefit from the capillary forces that may present. Thus, this interface can be referred to as a density-difference interface 115C. As an example, if fluid 120 is an oil, and fluid 130 is a gas, such as air, magnetizing particles can pass through the oil and into the air gap, and then move onto an elution fluid, a master mix fluid for nucleic acid processing, or some other similar downstream processing that may occur, which can provide additional contaminant clearing of the magnetizing particles and additional separation of fluid contamination between fluid 180 and fluid 140, for example. Thus, the capillary force gradient portion, if present, may include both a capillary force-supported interface and a density-difference interface.

In further detail, at the multi-fluid density gradient portions of the fluid column shown, the various fluids can be separated due to their density difference, and in the capillary force gradient portion, the various fluids can be separated by density as well, but may alternatively be separated by capillary forces in the form of providing the capillary force-supported interface, but there may also be density-difference interfaces within the capillary force gradient portion, as previously described.

The magnetic field generator 190 can be in the form of a movable magnet, or multiple magnets that provide the particle aggregating profile and/or the particle sweeping profile. Six columns are shown sequentially representing the same vertically layered fluid column at different points in time at (A-F) when different magnetic field profiles are being applied at different locations. At “A,” a particle aggregating profile is being used to apply a first magnetic field. Notably, the magnetizing particles are being concentrated or aggregated together near a side wall of the vessel. Aggregation can occur by applying an incubating magnetic field for from 1 second to 5 minutes, for example. At “B,” a particle sweeping profile is applied at a different location (either with the same magnet moved to a new location or by a different magnet similar to that shown in FIG. 1A). While this second magnetic field is being applied, the aggregated particles sweep across the first fluid 160 and become disaggregated. The same magnet, for example, can then be used to incubate the magnetizing particles for from 1 second to 5 minutes (or longer in some instances) to re-aggregate the particles, which corresponds to a third magnetic field (applied using a second particle aggregating profile—the particle aggregating profile may be different or the same as the one used previously, but as the magnet may have been moved to new locations, the magnetic field applied will be different relative to the column), as shown at “C.” Referring now to the column at (time) D, one of the magnets or a different magnet applies a fourth magnetic field to the column using a particle sweeping profile. The magnet, for example, can be moved downward to provide dynamic magnetic field and then can incubate with its magnetic field to sweep and disaggregate the magnetizing particles across the density-differential interface 115, e.g., from fluid 160 into fluid 170. A fifth magnetic field can be applied, as shown at “E,” where a third aggregated group of magnetizing particles is formed by application of the magnetic field using a particle aggregating profile. Regarding the column at (time) “F,” several potential additional magnetic fields are shown, alternating between application of particle aggregating profiles and particle sweeping profiles. Notably, in some instance, sweeping across the density-differential interfaces and/or the capillary force-supported interfaces 125 (if present) can occur without aggregating and/or without sweeping in some instances. However, in accordance with the present disclosure, magnetically moving the magnetizing particles is carried out using one or more particle aggregating profiles and one or more particle sweeping profiles. For example, there may be from 1 to 50, from 2 to 40, from 2 to 20, from 3 to 40, or from 4 to 50 magnetic field applications using a particle aggregating profile, and there may be from 1 to 50, from 2 to 40, from 2 to 20, from 3 to 40, or from 4 to 50 magnetic field applications using a particle sweeping profile.

With the examples shown in FIGS. 1-5, notably, there are other strategies that may be implemented to move the magnetizing particles along a positive or negative z-axis, including movement along the x- and y-axes as well when aggregating and/or sweeping. Examples of this are shown in FIGS. 1-5. However, aggregating and sweeping can be carried out in other ways, including though the use of permanent magnets and/or electrically induced magnetic elements, which can be at fixed positions or can be moveable about the column. Likewise, the column can be moved relative to the magnets. In another example, the magnet can be positioned adjacent to a side of the multi-fluid density gradient portion and can move vertically to cause the magnetizing particles to move therewith. In some examples, the magnet(s) can be moved along a side and/or along a bottom of the multi-fluid density gradient portion to pull the magnetizing particles in one direction or another. In one example, the magnet can be used to pull the magnetizing particles downwardly through fluid layers of the multi-fluid density gradient portion. In yet other examples, the magnet can be used to concentrate the magnetizing particles near a side wall of the multi-fluid density gradient portion to be moved downward by a movable magnet, or by a magnet positioned beneath the multi-fluid density gradient portion. In one example, a magnet used to move magnetizing particles downward can be used to reverse the direction of the magnetizing particles and can cause the magnetizing particles to re-enter a fluid layer that the magnetizing particles have previously passed through.

A strength of the magnetic field and the location of the magnet in relation to the magnetizing particles can affect a rate at which the magnetizing particles move downwardly through the multi-fluid density gradient portion. The further away the magnet and the lower the strength of the magnetic field, the slower the magnetizing particles will pass through the multi-fluid density gradient portion. In an example, a maximum distance between the magnet and a nearest location where one or more of the fluids resides along the multi-fluid density gradient portion can be about 50 mm maximum distance, about 40 mm maximum distance, about 30 mm maximum distance, about 20 mm maximum distance, or about 10 mm maximum distance. The minimum distance, on the other hand, may be from about 0.1 mm minimum distance, from about 1 mm minimum distance, or from about 5 mm minimum distance. In one example, the minimum distance between the magnet and the multi-fluid density gradient portion may be about the thickness of the vessel that contains the multi-fluid density gradient portion. Thus, distance ranges between the magnet and the multi-fluid density gradient portion can be from about 0.1 mm to about 50 mm (or more in some instances), from about 1 mm to about 50 mm, from about 1 mm to about 40 mm, from about 1 mm to about 30 mm, from about 1 mm to about 20 mm, from about 1 mm to about 10 mm, from about 5 mm to about 50 mm, or from about 5 mm to about 30 mm. In another example, a maximum distance between the magnet and a nearest location where one of the fluids resides along the multi-fluid density gradient portion can be about 30 mm.

In further detail regarding the vessels used, they can be configured as shown in the figures herein or can have other shapes. In one example, the vessel can include a conical chamber for a multi-fluid density gradient column or of a multi-fluid density gradient portion of vertically layered fluid column. If there is a capillary force gradient portion (with a capillary tube), that portion can be coupled to the large portion of the vessel and have a round cross section tube shape. Notably, either or both could include a round, square, triangle, rectangle, or other polygonal cross-section with an appropriate capillary junction. Either or both could include bifurcations or other structures within the vessel. The vessel may include one or more expansions and constrictions and/or may include a one-to-one, one-to-many, many-to-one, or many-to-many relationship between multi-fluid density gradient portions and capillary force gradient portions. The vessel may likewise include one or more input, output, or vent ports, and may or may not be symmetrical. Furthermore, the vessel can be made of various polymers (e.g. Polypropylene, TYGON, PTFE, COC, others), glass (e.g. borosilicate), metal (e.g. stainless steel), or a combination of materials. Additionally, the capillary component could be formed from multiple materials used in various microfluidic devices, such as silicon, glass, SU-8, PDMS, a glass slide, a molded fluidic channel(s), 3-D printed material, and/or cut/etched or otherwise formed features. Film layers can likewise be used for the structure as well. In further detail, the vessel may be monolithic or a combination of components fitted together. The vessel can be standalone or a component of a system (manual or automated) that includes functions or features for fluid positioning, particle manipulation, analysis, and/or other processes.

In further reference to the fluids used in the multi-fluid density gradient column (or portion of the column), can have a density that is altered using a densifier. Example densifiers can include sucrose, polysaccharides such as FICOLL™ (commercially available from Millipore Sigma (USA)), C₁₉H₂₆I₃N₃O₉ such as NYCODENZ® (commercially available from Progen Biotechnik GmbH (Germany)) or HISTODENZ™, iodixanols such as OPTIPREP™ (both commercially available from Millipore Sigma (USA)), or combinations thereof. In one example, a density difference of the first fluid layer relative to the second fluid layer can range from about 50 mg/mL to about 3 g/mL. In yet other examples, a density difference from the first fluid layer relative to the second fluid layer can range from about 50 mg/mL to about 500 mg/mL or from about 250 mg/mL to about 1 g/mL. In further detail, example additives that can be included in the first fluid layer, or in other fluid layers, depending on the design of the multi-fluid gradient column may include sucrose, C1-C4 alcohol, e.g., isopropyl alcohol, ethanol, etc., which can be included to adjust density, and/or to provide a function with respect to biological components or materials to pass through the column.

A quantity of fluid layers in the multi-fluid density gradient portion is not particularly limited. In one example, the multi-fluid density gradient portion can further include a fourth fluid layer having a fourth fluid density that can be greater than the third fluid density and can be positioned beneath the third fluid layer. The fourth fluid layer can be separated from the third fluid layer along a third fluid layer interface where the third fluid layer can be in fluid communication with the fourth fluid layer. In further examples, the assembly can further include a fifth, sixth, or seventh fluid layer that can be separated from the other fluids in the column based on a density of the fifth, sixth, or seventh fluid with respect to the other fluids in the column.

Any of the fluids along the column can be any of a number of combinations of fluids, such as gas fluid, e.g., air, aqueous fluid, non-polar fluid, polar, non-polar, miscible, or immiscible, etc. The fluids can be, for example a master mix fluid, reagent fluid, surface binding fluid, washing fluid, elution fluid, lysis fluid, etc. The fluids can likewise be pure, solutions, mixtures, suspensions, emulsions, and/or in other forms. They may or may not undergo chemical reactions within the vessel at any stage of the process, depending on the application. For example, a fluid in one layer can include a lysis buffer to lyse cells. In yet other examples, a fluid of another layer can be a surface binding fluid to bind the biological component to the magnetizing particles, a wash fluid to trap contaminants from a sample fluid and/or remove contaminants from an exterior surface of the magnetizing particles, a surfactant fluid to coat the magnetizing particles, a dye fluid, an elution fluid to remove the biological component from the magnetizing particles following extraction from the biological sample, a labeling fluid for binding labels to the biological component such as a fluorescent label (either attached to the magnetizing particles or unbound thereto), a reagent fluid to prep a biological component for further analysis such as a master mix fluid to prep a biological component for PCR, and so on.

In some examples, an individual fluid in one or multiple layers can provide sequential processing of a biological component from a biological sample. For example, individual fluids can carry out individual functions, and in many cases, the functions can be coordinated to achieve a specific result. Biological material that may be added can include whole blood, platelets, cells, lysed cells, cellular components, tissue, nucleic acids, e.g., DNA, RNA, primers, oligos, etc., or poly-bases, peptides, proteins, or the like. More specifically, for example, in considering biological material found in a cell, sequential fluid from top to bottom (as determined perpendicular to gravity or simulated centripetal force from a centrifuge) of a multi-fluid density gradient portion can act on the cell to lyse the cell in one of the fluids, and bind a target biological material from the lysed cell to magnetizing particles in a second fluid (or lysing and binding can alternatively be done in a single fluid). Additional fluid may be used to wash the magnetizing particles with the biological material bound thereto in another fluid, e.g., washing the second fluid from magnetizing particles in the next fluid, and/or eluting (or separating) the biological material from the magnetizing particles in yet another lower layer. The surface binding and cell lysis can occur, for example, with a lysate buffer in a sucrose and water solution, e.g., the lysate (lysis) buffer can be densified with sucrose. Washing can occur in a sucrose in water solution, for example. In other examples, one or more of the fluids can be present as a fluid (layer(s)) along the multi-fluid density gradient portion in the form of a master mix fluid for nucleic acid processing. Other combinations of fluids (first, second, third, etc.) may include a surface binding fluid, a washing fluid, and an elution fluid; or may include a lysis fluid, a washing fluid, a surface binding fluid, a second washing fluid, an elution fluid, and a reagent fluid. Regardless of the various functions of the various fluids with sequentially increasing densities arranged from top to bottom, at the individual fluids, the magnetizing particles can independently interact, e.g., become modified, with fluids as layers in order to sequentially process the magnetizing particles with surface active groups and/or biological material associated therewith or associated with one or more of the fluids, for example.

The term “associated,” “to associate with,” or the like, refers to an interaction between a surface of a magnetizing particle and a biological component of a biological sample. Thus, an association between the surface of the magnetizing particle and the biological component may refer to any type of attachment or adherence of a biological component with a surface of the magnetizing particle. This can include covalent bonding, electrostatic or ionic attraction, surface adsorption, hydrogen bonding, and/or other adherence or linkage suitable for moving biological component together with the particulate substrate. In accordance with this, in some examples, the biological component separator can include a biological sample and the magnetizing particles can be surface activated to preferentially bind with a biological component relative to secondary components in a biological fluid sample. In some examples, the magnetizing particles can include ligands attached thereto, such as proteins, antibodies, antigens, nucleic acid primers, amino groups, carboxyl groups, epoxy groups, tosyl groups, sulphydryl groups, or a combination thereof, or the like. Regarding combinations of ligands, there can be multiple types of ligands on a common magnetizing particle, a mixture of magnetizing particles with different ligands on multiple portions of the magnetizing particles (with the same or different magnetizing particles). The ligand can be selected to correspond with and bind with the biological component and can vary based on the type of biological component being isolated from the biological sample. For example, the ligand can include a nucleic acid primer when isolating a biological component that includes a nucleic acid sequence. In another example, the ligand can include an antibody when isolating a biological component that includes an antigen. By way of example, commercially available examples of magnetizing particles with surface-activated groups include those sold under the trade name DYNABEADS®, available from ThermoFischer Scientific (USA).

A vertical height of the various layers of fluids in the multi-fluid density gradient portion can vary. Adjusting a vertical height of a fluid layer can affect a residence time of the paramagnetizing particles in that fluid layer. The taller the fluid layer, in some instances, there may be a longer the residence time of the magnetizing particles in the fluid layer. However, this could depend on the rate at which the magnetizing particles fall or are drawn through the column. In some examples, all of the fluid layers in the multi-fluid density gradient portion can be the same vertical height. In other examples, a vertical height of individual fluid layers in a multi-fluid density gradient portion can vary from one fluid layer to the next. In one example, a vertical height of the various layers along the multi-fluid density gradient portion can individually range from about 10 μm to about 50 mm. In another example, a vertical height of the fluid layers along the multi-fluid density gradient portion can individually range from about 10 μm to about 30 mm, from about 25 μm to about 1 mm, from about 200 μm to about 800 μm, or from about 1 mm to about 50 mm.

In further detail regarding the magnetizing particles, these particles can include surfaces that are associated with a biological component or can be formulated to become associated with a biological component in situ. Alternatively, the system can include a biological sample and the magnetizing particles (magnetizing particles or otherwise) can be surface-activated to preferentially bind with a biological component relative to secondary components in a biological fluid sample. Thus, the biological component may preferentially bind to the surface compared to secondary components such as enzymes, cellular debris, lysing agents, buffers, or a combination thereof. The magnetizing particles can be loaded in any of the fluid layers or preloaded with biological component bound to surface of the magnetizing particles to be then dispersed in one or more of the fluids. Once in the multi-fluid density gradient column, the magnetizing particles can be moved vertically in either direction (up or down) using magnetic fields applied from both a particle aggregating profile and at particle sweeping profile. Movement in the x- and/or y-axes can also occur when aggregating and/or sweeping the magnetizing particles.

In further detail regarding the magnetizing particles, the magnetizing particles can be particles with a density suitable for gravity settling or centrifugation separation or movement along the column in a negative z-direction or may be buoyant to promote movement in a positive z-direction. Thus, when not acted upon with magnetic field, they can still move within the fluids of the column in some instances.

The magnetizing particles can be in the form of paramagnetizing particles, superparamagnetizing particles, diamagnetizing particles, or a combination thereof, for example. Whether using magnetizing particles or otherwise, particle surfaces can be surface-activated or can have surface chemistry that is inherent in becoming associated with a biological component to bind with a biological component of interest or bind to a biological component that is to be removed from a fluid with the biological component of interest. This association with a biological component can be as described and defined above.

In some examples, the magnetizing particles can have an average particle size that can range from about 0.1 μm to about 70 μm. The term “average particle size” describes a diameter or average diameter, which may vary, depending upon the morphology of the individual particle. A shape of the magnetizing particles can be spherical, irregular spherical, rounded, semi-rounded, discoidal, angular, sub-angular, cubic, cylindrical, or any combination thereof. In one example, the particles can include spherical particles, irregular spherical particles, or rounded particles. The shape of the magnetizing particles can be spherical and uniform, which can be defined herein as spherical or near-spherical, e.g., having a sphericity of >0.84. Thus, any individual particles having a sphericity of <0.84 are considered non-spherical (irregularly shaped). The particle size of the substantially spherical particle may be provided by its diameter, and the particle size of a non-spherical particle may be provided by its average diameter (e.g., the average of multiple dimensions across the particle) or by an effective diameter, e.g., the diameter of a sphere with the same mass and density as the non-spherical particle. In further examples, the average particle size of the magnetizing particles can range from about 1 μm to about 50 μm, from about 5 μm to about 25 μm, from about 0.1 μm to about 30 μm, from about 40 μm to about 60 μm, or from about 25 μm to about 50 μm.

In an example, the magnetizing particles can be unbound to a biological component when added directly to one of the fluid (layers) of a multi-fluid density gradient portion. Binding between the magnetizing particles and the biological component of the biological sample can occur in the multi-fluid density gradient portion. In yet another example, the magnetizing particles and a biological sample including a biological component can be combined in a loading fluid before being added to a multi-fluid density gradient portion. In this example, binding of the magnetizing particles to the biological component of the biological sample can occur in the multi-fluid density gradient portion.

With more specific detail regarding the magnetizing particles, the term “magnetizing particles” is defined herein to include microparticles that may not be magnetic in nature unless and until a magnetic field is introduced at a strength and proximity to cause them to become magnetic. Their magnetic strength can be dependent on the magnetic field applied and may get stronger as the magnetic field is increased, or the magnetizing particles get closer to the magnetic source that is applying the magnetic field. In more specific detail, “paramagnetizing particles” have these properties, in that they have the ability to increase in magnetism when a magnetic field is present; however, paramagnetizing particles are not particularly magnetic when a magnetic field is not present. In some examples, the paramagnetizing particles can exhibit no residual magnetism once the magnetic field is removed. A strength of magnetism of the paramagnetizing particles can depend on the strength of the magnetic field, the distance between a source of the magnetic field and the paramagnetizing particles, and a size of the paramagnetizing particles. As a strength of the magnetic field increases and/or a size of the paramagnetizing particles increases, the strength of the magnetism of the paramagnetizing particles increases. As a distance between a source of the magnetic field and the paramagnetizing particles increases the strength of the magnetism of the paramagnetizing particles decreases. “Superparamagnetizing particles” can act similar to paramagnetizing particles; however, they can exhibit magnetic susceptibility to a greater extent than paramagnetizing particles in that the time it takes to become magnetized appears to be near zero seconds. “Diamagnetizing particles,” on the other hand, can display magnetism due to a change in the orbital motion of electrons in the presence of a magnetic field.

### Methods of Separating Biological Components

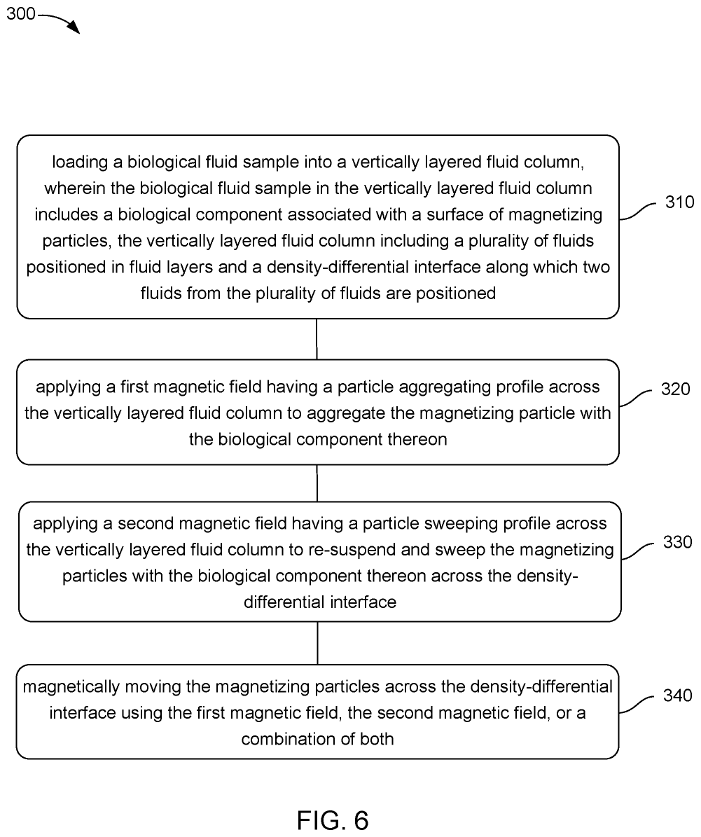

A flow diagram 300 of a method of separating a biological component from a biological sample is shown in FIG. 6. The method can include loading 310 a biological fluid sample into a vertically layered fluid column, wherein the biological fluid sample in the vertically layered fluid column includes a biological component associated with a surface of magnetizing particles. The vertically layered fluid column can include a plurality of fluids positioned in fluid layers and a density-differential interface along which two fluids from the plurality of fluids are positioned. The method further includes applying 320 a first magnetic field having a particle aggregating profile across the vertically layered fluid column to aggregate the magnetizing particle with the biological component thereon, and applying 330 a second magnetic field having a particle sweeping profile across the vertically layered fluid column to re-suspend and sweep the magnetizing particles with the biological component thereon across the density-differential interface. The method further includes magnetically moving 340 the magnetizing particles across the density-differential interface using the first magnetic field, the second magnetic field, or a combination of both. In one example, the biological component may be a nucleic acid, and the biological sample includes secondary components that are not associated with the surface of the magnetizing particles, e.g., secondary components can be enzymes, cellular debris, lysing agents, buffers, or a combination thereof. In another example, the method can further include applying a third magnetic field having a particle aggregating profile that is different than the particle aggregating profile of the first magnetic field, and also applying a fourth magnetic field having a particle sweeping profile across that is different than the particle sweeping profile of the second magnetic field. The particle aggregating profile can include, for example, an incubation time ranging from 1 second to 5 minutes (where the magnetic field is held static so the particles can collect and aggregate). The sweeping profile can also include an incubation time ranging from 1 second to 5 minutes and/or may include a dynamically changing magnetic field (where the magnetic field is modified, such as by magnet movement, modified electromagnetic energy applied to electrically induced magnetic elements, etc.

In some other examples, the biological sample including the biological component can be combined with the magnetizing particles in a loading solution prior to loading the biological sample including the biological component and the magnetizing particles into the multi-fluid density gradient portion. For example, the magnetizing particles and the biological sample can be admixed in a loading fluid. The biological sample and the magnetizing particles can be permitted to incubate or otherwise become prepared for loading on top of or into the multi-fluid density gradient portion. The magnetizing particles can bind with the biological component in the loading fluid and can then be added to the multi-fluid density gradient portion for the fluid layers to act upon the magnetizing particles. In one example, the loading fluid can become the uppermost fluid layer when loading from the top or can become the lowermost fluid layer when loading from the bottom, for example. Other fluid layers beneath or above the loading layer can be included through which the magnetizing particles is passed in part or in full.

The fluid used for loading the column (or the first fluid, or even the second fluid, or other fluid layer) can include secondary components selected from enzymes, cellular debris, lysing agents, buffers, or a combination thereof. The magnetizing particles can be bound to the biological component in a loading fluid or in a subsequent fluid along the multi-fluid density gradient portion. In the case of a loading fluid, magnetizing particles including the biological component bound thereto can then be introduced as a separate fluid layer for the magnetizing particles to be drawn into other fluid layers that can act on the microfluidic particles to further interact with the surface thereof along the multi-fluid density gradient portion.

In accordance with the method, the magnetizing particles can be passed through multiple density-differential interfaces, and in some instances, a capillary force-supported interface, depending on the arrangement. The magnetizing particles can be passed through any or all of these interfaces from fluid to fluid in an upward or downward z-axis direction, though movement along the x- and y-axes also typically occurs during sweeping and aggregating.

In one example, the method can further include selectively withdrawing, e.g., pipetting, the biological component out of the third fluid layer, such as through an ingress/egress opening(s) from the top, the bottom, or through a sidewall, for example. The biological component may still be associated with a surface of the magnetizing particles or may be separated from the magnetizing particles. In another example, this method alternatively may include selectively withdrawing, e.g., pipetting, from one of the fluids (one of the layers), the second fluid layer, and/or the third fluid layer out of the multi-fluid density gradient portion and leaving the magnetizing particles with the biological component bound thereto in a vessel of the multi-fluid density gradient portion to either be further treated or removed after the extraction of one of the fluids, the second fluid layer, and the third fluid layer therefrom. In some examples, the biological sample can include a cell with the biological component trapped within the cell (prior to lysis), a virus, or a biological component with extra-cellular vesicles. Lysing the cell can release the biological component therefrom and can permit isolation of the biological component. In this example, one of the fluids (or a loading fluid) can include a lysing agent for the cell. The method can further include lysing the cell in situ within one of the fluids or the loading fluid so that the biological component can be liberated from the cell and can bind with the magnetizing particles in one of the fluids (or fluid layers) or the loading fluid.

As another example, the moving the magnetizing particles through the biological component separators can be carried out using any of a number of fluids in fluid layers, which can be layers of gas fluid, e.g., air, aqueous fluid, non-aqueous fluid, non-polar fluid, master mix fluid, reagent fluid, surface binding fluid, washing fluid, elution fluid, lysis fluid, etc. To illustrate, in a first fluid layer, lysing and particle binding may occur as cells, viral particles, or the like and are initially lysed and a nucleic acid is released into the first fluid. The nucleic acid can become bound to a surface of the particles, which can be magnetizing particles. Next, as the particles are drawn through a density-differential interface or a capillary force-supported interface and into a second fluid, cellular debris and unbound nucleic acid can be exchanged for a wash buffer fluid. A second wash can occur at a third fluid layer as the bead-bound nucleic acid is further cleared of contaminants, e.g., lysed cellular debris and/or other contaminants or other material not of interest that may be present. Next, in some instances, oil exclusion may be carried out so that aqueous solution entrapped in the magnetizing particles can be replaced with oil, e.g., mineral oil. Other fluids may be suitable for this, but mineral oil is a good example of a fluid that may benefit from separation due to capillary forces in accordance with the present disclosure. Furthermore, by using an oil, this can provide an effective way of transitioning the magnetizing particles from being carried by a liquid fluid and passed into a gaseous fluid, such as at an air layer. Thus, the air layer or gap can be used to clear the contaminants further and can provide a mechanism to provide little to no contact between the fluids about the air gap. Magnetizing particles with entrapped mineral oil can be pulled into the air gap, providing reduced likelihood of concentration of contact between wash and/or lysis buffer and the next liquid beneath the air gap, which can be, for example, an elution buffer, a master mix fluid for nucleic acid processing, or the like, for example. Other example processing sequences can likewise be used in accordance with the present disclosure.

To provide further example detail regarding how a vertically layered fluid column may be used to process a biological component, for example, a biological component separation process can be carried out using a biological component separator as shown in FIG. 5. For example, a biolgoicla material including cells, viral particles, or the like may be initially lysed and a nucleic acid 220 is released in fluid 160. The lysis-binding in fluid 180, for example, and can be carried out using a solution including one or more of:
guanidine salt-based or other high salt content buffers used for solid phase extraction;
alcohol such as isopropyl alcohol (IPA), ethanol (EtOH), polyethylene glycol (PEG), or other suitable alcohols;
carrier nucleic acid(s);
enzymes to assist in lysis, such as Proteinase K, for example; and/or
pH adjuster to modify pH.

The nucleic acid from the cell can become bound to a surface of the magnetizing particles 110. Next, as the particles are drawn through interface 115A, cellular debris and unbound nucleic acid can be exchanged for a wash buffer at fluid 170. A second wash can occur at fluid 180, as bead-bound nucleic acid is further cleared of contaminants, e.g., lysed cellular debris and/or other contaminants or other material not of interest that may be present. Example wash buffers for use at fluids 170 and/or 180 can be:
an aqueous solution including an alcohol, such as ethanol, and one or more of another alcohol, a binding agent binding agent, a salt, a surfactant, and/or a stabilizing agent; and/or
MyOne™ silane genomic DNA or viral kits, mRNA Direct kits, Mag Max™ kits, or other similar kits that include wash buffers often used with DYNABEADS®, all available from ThermoFischer Scientific, USA.

In further detail regarding, the capillary force gradient portion of the vertically layered fluid column, in this example, there are two fluids that can work together to clear debris from a particulate substrate as it is moved from the multi-fluid density gradient column portion (after the two layers of wash buffer) and into the capillary force gradient portion of the column. Those two fluids include the use of an oil (which can be present at fluid 120) and a gas (which can be present at fluid 130). The oil can be, for example, mineral oil. The gas can be, for example, air. Thus, the oil can be used for oil exclusion, which is shown as being carried out at (D), where aqueous solution that may be present on or even entrapped in the particulate substrate (or magnetizing particles) can be replaced with the oil. Other fluids may be suitable for this, but mineral oil is a good example of a fluid that may benefit from separation due to capillary forces in accordance with the present disclosure. Furthermore, by using an oil, this can provide an effective way of transitioning the particulate substrate from being carried by a liquid fluid and passed into a gaseous fluid, such as air.

Regarding the oil layer in the capillary force gradient portion of the column, shown at (D), specific oils that can be used include:
light oils, such as mineral oil for molecular biology or molecular grade mineral oils, light oil M5904 (density 0.84 g/mL at 25° C.) from Sigma-Aldrich (USA);
olive oil, such as high purity olive oil; and/or
densified oil.

Once the particulate substrate passes through the oil (fluid 120), a gas (fluid 130), which in this instance can be air (or an air gap), can be used to clear the contaminants further and can provide a mechanism to provide little to no contact between the fluids above and below the air gap. The biological component being separated (or further processed) has now have been loaded on the particulate substrate after cell lysis, washed in two different wash buffer layers, further contaminant-cleared by the oil, and passed through the air gap, providing reduced likelihood of concentration of contact between wash and/or lysis buffer and the next fluid beneath the air gap, e.g., elution buffer, a master mix fluid for nucleic acid processing, or the like. To separate the biological component, e.g., nucleic acid, from the particulate substrate, an elution buffer can be used. Example elution buffers suitable for use may include one or more of:
aqueous salt solution (sufficient for elution but to retain biological component intact);
stabilizers;
surfactants; and/or
master mix if it is for a direct elution process, provide column is tuned for a target biological component of interest.

### Definitions

It is noted that, as used in this specification and the appended claims, the singular forms “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and determined based on experience and the associated description herein.

As used herein, the phrase “in fluid communication” or “in direct contact” indicates that two or more fluids are fluidly coupled to one another, either directly or in some instances with intervening fluid(s) therebetween. In accordance with this definition, the term “in fluid communication” excludes fluids that are separate by a physical barrier, but rather are separated by density or using capillary forces as described herein, for example.

As used herein, the term “interact” or “interaction” as it relates to a surface of the magnetizing particles indicates that a chemical, physical, or electrical interaction occurs where a magnetizing microparticle surface property is modified in some manner that is different than may have been present prior to entering the fluid layer, but does not include modification of magnetic properties magnetizing particles as they are influenced by the magnetic field introduced by the magnet. For example, a fluid layer can include a lysis buffer to lyse cells, and cellular components can become associated with a surface of the magnetizing particles. Lysing cells in a fluid can modify the fluid sample and thus modify or interact with a surface of magnetizing particles, e.g., the cellular component binds or becomes associated with a surface of the magnetizing particles. In yet other examples, a fluid layer that would be considered to interact with the magnetizing particles could be a wash fluid layer to trap contaminates from a sample fluid and/or remove contaminates from an exterior surface of the magnetizing particles, a surfactant fluid layer to coat the magnetizing particles, a dye fluid layer to introduce visible or other markers to the fluid or surface, an elution fluid layer to remove the biological component from the magnetizing particles following extraction from the biological sample, a labeling fluid layer for binding labels to the biological component such as a fluorescent label (either attached to the magnetizing particles or unbound thereto), a reagent fluid layer to prep a biological component for further analysis such as a master mix fluid layer to prep a biological component for PCR, and so on.

As used herein, a plurality of items, structural elements, compositional elements, and/or materials may be presented in a common list for convenience. However, these lists should be construed as though individual members of the list is individually identified as a separate and unique member. Thus, no individual member of such list should be construed as a de facto equivalent of any other member of the same list solely based on presentation in a common group without indications to the contrary.

Concentrations, amounts, and other numerical data may be expressed or presented herein in a range format. A range format is used merely for convenience and brevity and thus should be interpreted flexibly to include not only the numerical values explicitly recited as the limits of the range, but also to include individual numerical values or sub-ranges encompassed within that range as if numerical values and sub-ranges are explicitly recited. As an illustration, a numerical range of “about 1 wt % to about 5 wt %” should be interpreted to include not only the explicitly recited values of about 1 wt % to about 5 wt %, but also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting one numerical value. Furthermore, such an interpretation should apply regardless of the breadth of the range or the characteristics being described.

While the present technology has been described with reference to certain examples, various modifications, changes, omissions, and substitutions can be made without departing from the spirit of the disclosure. The disclosure is limited only by the scope of the following claims.

What is claimed is:

1. A biological component separator, comprising:
a vertically layered fluid column, including a plurality of fluids positioned in fluid layers and a density-differential interface along which two fluids from the plurality of fluids are positioned, and
a magnetic field generator positioned about the vertically layered fluid column and having multiple magnetic field profiles appliable across the vertically layered fluid column, wherein the multiple magnetic field profiles including a particle aggregating profile to concentrate magnetizing particles when present in the vertically layered fluid column and a particle sweeping profile to re-suspend and sweep the magnetizing particles when present across the vertically layered fluid column.

2. The A biological component separator of claim 1, wherein a density difference of the first fluid relative to the second fluid is from about 50 mg/mL to about 3 g/mL.

3. The biological component separator of claim 1, wherein the particle aggregating profile also causes the magnetizing particles to move across the density-differential interface along a z-axis of the vertically layered fluid column.

4. The biological component separator of claim 1, wherein the particle sweeping profile includes a dynamically changing magnetic field that causes magnetizing particles to move along a z-axis of the vertically layered fluid column and across the density-differential interface.

5. The biological component separator of claim 1, wherein the magnetic field generator includes one or multiple permanent magnets, wherein the one or more permanent magnets have a first spatial relationship relative to the vertically layered fluid column to apply the particle aggregating profile and a second spatial relationship relative to the vertically layered fluid column to apply the particle sweeping profile.

6. The biological component separator of claim 1, wherein the magnetic field generator includes one or multiple electrically induced magnetic elements, the particle aggregating profile and particle sweeping profile are different as a result of a different electrical profile applied to the one or multiple electrically induced magnetic elements.

7. A biological component separation system, comprising:
a vertically layered fluid column, including a plurality of fluids positioned in fluid layers and a density-differential interface along which two fluids from the plurality of fluids are positioned;
magnetizing particles including particle surfaces to associate with a biological component of a biological fluid sample; and
a magnetic field generator positioned about the vertically layered fluid column and having multiple magnetic field profiles appliable across the vertically layered fluid column, wherein the multiple magnetic field profiles including a particle aggregating profile to concentrate the magnetizing particles and a particle sweeping profile to re-suspend and sweep the magnetizing particles across the vertically layered fluid column.

8. The biological component separation system of claim 7, further comprising the biological fluid sample, wherein the magnetizing particles are associated with the biological component relative to secondary components in the biological fluid sample.

9. The biological component separation system of claim 7, wherein the biological component is a nucleic acid and the magnetizing particles are surface-activated for positive isolation of the nucleic acid to separate the nucleic acid from secondary components.

10. The biological component separation system of claim 7, wherein the biological component is a secondary component that is other than a nucleic acid and the magnetizing particles are surface-activated for negative isolation to separate the nucleic acid from the secondary component.

11. The biological component separation system of claim 7, wherein the particle aggregating profile incudes an incubation period, and wherein the particle sweeping profile includes a dynamically changing magnetic field, wherein one or both of the particle aggregating profile or the particle sweeping profile causes magnetizing particles to move along a z-axis of the vertically layered fluid column and across the density-differential interface.

12. A method of separating a biological component from a biological fluid sample, comprising:
loading a biological fluid sample into a vertically layered fluid column, wherein the biological fluid sample in the vertically layered fluid column includes a biological component associated with a surface of magnetizing particles, the vertically layered fluid column including a plurality of fluids positioned in fluid layers and a density-differential interface along which two fluids from the plurality of fluids are positioned;
applying a first magnetic field having a particle aggregating profile across the vertically layered fluid column to aggregate the magnetizing particle with the biological component thereon;
applying a second magnetic field having a particle sweeping profile across the vertically layered fluid column to re-suspend and sweep the magnetizing particles with the biological component thereon across the density-differential interface; and
magnetically moving the magnetizing particles across the density-differential interface using the first magnetic field, the second magnetic field, or a combination of both.

13. The method of claim 12, wherein the biological component is a nucleic acid, and wherein the biological sample includes secondary components that are not associated with the surface of the magnetizing particles, the secondary components selected from enzymes, cellular debris, lysing agents, buffers, or a combination thereof.

14. The method of claim 12, wherein the further comprising:
applying a third magnetic field having a particle aggregating profile that is different than the particle aggregating profile of the first magnetic field; and
applying a fourth magnetic field having a particle sweeping profile across that is different than the particle sweeping profile of the second magnetic field.

15. The method of claim 12, wherein the particle aggregating profile includes incubation time ranging from 1 second to 5 minutes, and wherein the sweeping profile includes an incubation time ranging from 1 second to 5 minutes, a dynamically changing magnetic field, or a combination of both.
